(19) United States
(12) Patent Application Publication
(10) Pub. No.: US 2017/0172959 A1
(43) Pub. Date: Jun. 22, 2017

(54) D-SERINE FOR THE TREATMENT OF VISUAL SYSTEM DISORDERS

(71) Applicant: Allergan, Inc., Irvine, CA (US)

(72) Inventors: Yong-Xin Li, Mission Viejo, CA (US); Alan C. Foster, San Diego, CA (US); Ursula V. Staubli, Laguna Beach, CA (US); John E. Donello, Dana Point, CA (US); Miguel Alcantara, Aliso Viejo, CA (US)

(21) Appl. No.: 15/220,003

(22) Filed: Jul. 26, 2016

Related U.S. Application Data
(63) Continuation of application No. 13/523,579, filed on Jun. 14, 2012, now abandoned.
(60) Provisional application No. 61/498,186, filed on Jun. 17, 2011.

(51) Int. Cl. A61K 31/198; A61K 31/42; A61K 31/4015

(52) U.S. Cl. CPC A61K 31/198; A61K 31/4015; A61K 31/42

(57) ABSTRACT

The present invention relates to therapeutic methods using pharmaceutical compositions comprising one or a combination of co-agonists at the N-methyl-D-aspartate receptor for the treatment of visual system disorders.

### CROSS REFERENCE TO RELATED APPLICATIONS

This application is a continuation of U.S. patent application Ser. No. 13/523,579, filed Jun. 14, 2012, which claims the benefit of U.S. Provisional Application Ser. No. 61/498,186 filed Jun. 17, 2011, the disclosures of which are hereby incorporated by reference in their entireties and serve as the basis of a priority and/or benefit claim for the present application.

### FIELD OF THE INVENTION

The present invention relates to therapeutic methods using pharmaceutical compositions comprising co-agonists at the N-methyl-D-aspartate receptor for the treatment of visual system disorders.

### SUMMARY OF THE RELATED ART

The N-methyl-D-aspartate (NMDA) sub-type of glutamate receptors plays a key role in the central nervous system (CNS) including the visual function. It has been suggested that retinal ganglion cell AMPA and NMDA receptors play complementary roles in synaptic transmission and that NMDA receptors contribute to visual contrast coding (Manookin et al., 2010). NMDA receptors have been shown to be important for the development and refinement of the correct topographic projections from retinal ganglion cell (RGCs) to the superior colliculus (Simon et al., 1992; Shi et al., 1997). In visual cortex, NMDA receptor-mediated plasticity is involved in the recovery of cortical binocularity from the effects of monocular deprivation (MO). NMDA receptors are ligand-gated cation channels comprised of a tetrameric assembly of NR1, NR2 and NR3 sub-units (Paoletti and Neyton, 2007). They are unique amongst neurotransmitter receptors in that they require occupation of two separate recognition sites for activation. An acidic amino acid site where glutamate binds is located on the NR2 sub-units, and a neutral amino acid (or co-agonist) site is located on the NR1 sub-unit. The endogenous co-agonist for this site was originally thought to be glycine, but more recent evidence indicated that D-serine is also an endogenous co-agonist. In fact, in higher brain regions D-serine may be the dominant co-agonist (Mothet et al, 2000). Occupation of the co-agonist site is essential for glutamate (or a glutamate analog) to activate the NMDA receptor, and in native assays the removal of glycine or D-serine by exogenously-applied degradative enzymes can reduce or abolish NMDA receptor-mediated responses. In addition to glycine and D-serine several additional co-agonists have been identified: D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966, 1-amino-1-carboxycyclobutane.

These are classed as “full co-agonists” having the same maximal efficacy as the endogenous co-agonists glycine and D-serine, or as “partial co-agonists” that can increase NMDA receptor responses, but with a maximal efficacy that is less than the endogenous co-agonists.

Endogenous D-serine contributes to NMDA-receptor-mediated light-evoked responses in the vertebrate retina (Gustafson et al, 2007) and light-evoked NMDA receptor-mediated currents are reduced by blocking D-serine synthesis in retina (Stevens et al, 2010). NMDA receptors have also been shown to mediate synaptic responses in the lateral geniculate (Harveit & Heggelund, 1990; Scharfman et al., 1990) and the visual cortex (ie the primary pathways that transduce visual information). In the visual cortex, NMDA receptors mediate the phenomenon of long-term potentiation (LTP), an important form of synaptic plasticity. NMDA receptor-dependent LTP occurs in many brain regions and is viewed as a mechanism of synaptic strengthening that is fundamental to the establishment and maintenance of appropriate synaptic connections. In the hippocampus, for example, LTP has been studied as a synaptic surrogate of learning and memory. In visual cortex neurons, LTP mediates stimulus-specific response potentiation, a form of experience-dependent plasticity that contributes to visual function (Cooke and Bear, 2010).

### BRIEF DESCRIPTION OF THE DRAWINGS

FIG. 1 represents a graph showing that D-serine dose-dependently facilitates long-term potentiation (LTP) in the primary visual cortex of rats.

FIG. 2 shows the experimental data collected in rats eighteen weeks after blue-light treatment. Filled squares represent the contrast sensitivities measured from control rats. Dots represent the contrast sensitivities measured from rats eighteen weeks after blue-light treatment.

FIG. 3 shows that D-serine improved the contrast sensitivity impaired in Long-Evans rats by blue-light treatment. The experiments were performed sixteen (for 600 mg/kg) and fifty weeks (for 100 mg/kg) after blue-light treatment. Saline was used as a control. Half of the rats were injected with 100 or 600 mg/kg D-serine and the other half with saline in the first test; cross-over exposure took place one week later; the spatial frequencies were fixed at 0.575 cpd.

### DETAILED DESCRIPTION OF THE INVENTION

In one aspect, the present invention relates to a method for the treatment of visual system disorders, the method comprising administering to a subject in need thereof an ophthalmically acceptable pharmaceutical composition containing a therapeutically effective amount of one or a combination of co-agonist(s) at the NMDA receptor.

In another aspect, the present invention relates to a method wherein the co-agonist at the NMDA receptor is selected from: Glycine, D-Serine, D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966, 1-amino-1-carboxycyclobutane.

In another aspect, the present invention relates to a method, wherein the pharmaceutical composition contains a therapeutically effective amount of D-serine.

In another aspect, the present invention relates to a pharmaceutical composition comprising as active ingredient a therapeutically effective amount of one or a combination of co-agonist(s) at the NMDA receptor and a pharmaceutically acceptable adjuvant, diluents or carrier.

In another aspect, the present invention relates to a pharmaceutical composition, wherein the active ingredient is one or a combination of co-agonist(s) at the NMDA receptor.

In another aspect, the present invention relates to a pharmaceutical composition, wherein the co-agonist at the NMDA receptor is selected from Glycine, D-Serine, D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966, 1-amino-1-carboxycyclobutane.

In another aspect, the present invention relates to a pharmaceutical composition, wherein the active ingredient is D-serine.

Visual system disorders which may be treated with the D-serine transport inhibitors include macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigement epitheliitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis, glaucoma, amblyopia, stroke-induced blindness, visual system disorder in Parkinson's disease, Alzheimer's disease and multiple sclerosis, seizure-induced cortical blindness, induced visual system disorder, and epileptic blindness.

In another aspect the present invention relates to a method for the treatment of visual system disorders caused by a deficit in N-methyl-D-aspartate receptor function, the method comprising administering to a subject in need thereof an ophthalmically acceptable pharmaceutical composition containing a therapeutically effective amount of D-serine.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back of the eye, front of the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable to preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Invention compounds may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

An opthalmically acceptable pharmaceutical composition is one that can be administered topically to the eye of a subject in need thereof. Comfort to the subject being administered the composition should be maximized, but other considerations, such as drug stability, may necessitate a pharmaceutical composition that provides less than optimal comfort. In such a case, the composition should be formulated such that it is tolerable to a subject being administered the composition topically.

The claimed pharmaceutical composition can be administered topically in the form of solutions or suspensions, ointments, gels, creams, etc. A “pharmaceutically acceptable excipient” is one that is compatible with the active ingredient of the composition and not harmful to the subject being administered the pharmaceutical composition. Solutions for ophthalmic application are often prepared using physiological saline as a major vehicle. Other vehicles include polyvinyl alcohol, povidone, hydroxypropyl methyl cellulose, poloxamers, carboxymethyl cellulose, hydroxyethyl cellulose, and purified water. Examples of useful excipients also include preservatives, buffers, other pH adjustors, tonicity adjustors, surfactants, antioxidants, and chelating agents.

Useful preservatives include benzalkonium chloride, chlorobutanol, thimerosal, phenylmercuric acetate and phenylmercuric nitrate. Examples of buffers include phosphate, borate, sulfate, acetate, and citrate buffers. Acids or bases may be used to adjust the pH of the compositions as needed. Examples of tonicity agents include glycerin, mannitol, sodium chloride and potassium chloride. Useful surfactants include, for example, Tween 80. Examples of ophthalmically acceptable antioxidants include sodium metabisulfite, sodium thiosulfate, acetylcysteine, butylated hydroxyanisole and butylated hydroxytoluene. A useful chelating agent is edentate disodium.

Mixtures of two or more of any suitable excipients may be used.

Aside from topical application to treat diseases affecting the eye including glaucoma, pharmaceutical compositions containing at least one compound of formula (I) can also be administered periocularly, intraocularly, or by other effective means available in the art.

Persons skilled in the art would readily understand that a drug containing one or more of the compounds disclosed herein can be confected as a powder, pill, tablet or the like, or as a solution, emulsion, suspension, aerosol, syrup or elixir suitable for oral or parenteral administration or inhalation. For solid dosage forms or medicaments, non-toxic solid excipients for admixture with compounds disclosed herein include, but are not limited to, pharmaceutical grades of mannitol, lactose, starch, magnesium stearate, sodium saccharin, polyalkylene glycols, talcum, cellulose, glucose, sucrose, and magnesium carbonate. The solid dosage forms may be coated by a material such as glyceryl monostearate or glyceryl distearate, which is utilized in known techniques to delay disintegration and absorption in the gastrointestinal tract for the purpose of providing a sustained action over a longer period. Solid dosage forms may also be coated by the techniques described in U.S. Pat. Nos. 4,256,108, 4,166,452 and 4,265,874 to form osmotic therapeutic tablets for control release.

Pharmaceutically administrable liquid dosage forms can, for example, comprise a solution or suspension of at least one of the compounds disclosed herein and optional pharmaceutical adjutants in a carrier, such as water, saline, aqueous dextrose, glycerol, ethanol and the like. The liquid dosage forms may also contain nontoxic auxiliary substances such as wetting or emulsifying agents, pH buffering agents and the like. Examples of such auxiliary agents include sodium acetate, sorbitan monolaurate, triethanolamine, sodium acetate, triethanolamine oleate, etc. Methods for preparing such dosage forms are well-known to persons skilled in the art (see, for example, Reminton's Pharmaceutical Sciences, Mack Publishing Company, Easton, Pa., 16^{th} Edition, 1980).

Parenteral administration is generally characterized by subcutaneous, intramuscular, or intravenous injection. Injectables can be prepared as liquid solutions or suspensions, solid forms that can be reconstituted into solutions or suspensions prior to injection, or as emulsions. Suitable excipients include water, saline dextrose, glycerol, ethanol and the like. Such injectable pharmaceutical compositions may also contain minor amounts of non-toxic auxiliary substances such as wetting or emulsifying agents, pH buffers and the like.

Examples mentioned herein are not intended to limit the scope of the invention in any way.

The effects of D-serine on LTP in visual cortex

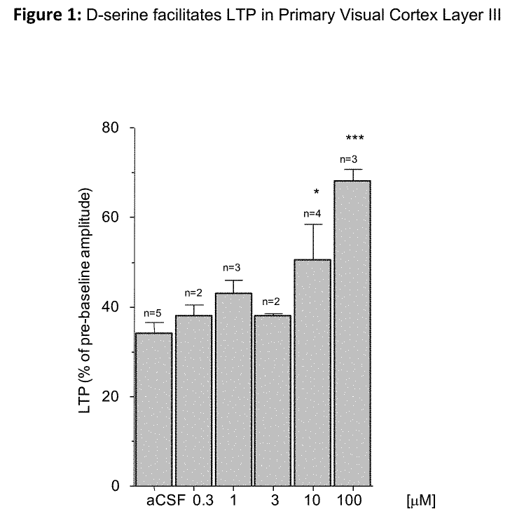

It was found that exogenous D-serine significantly enhanced the LTP in the primary visual cortex of adult rats (FIG. 1). Ito and Hicks (Neuroscience Letters 303 (2001) 95-98) report that D-serine alone did not enhance LTP in slices of kitten visual cortex and only produced a reversal of the suppressant effects of the co-agonist site antagonist, 7-chlorokynurenic acid.

The effects of D-serine on contrast sensitivity in rats with retinal damage induced by blue-light exposure.

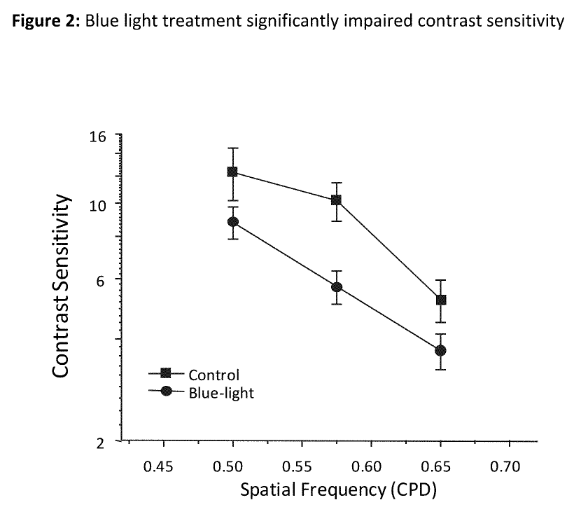

Blue-light treatment damages photoreceptors in the retina, and has been proposed as a model of age related macular degeneration (ARMID, Wielgus et al., 2010). In blue-light treated Long-Evans rats, contrast sensitivity, an important measure of visual performance, was significantly impaired (FIG. 2).

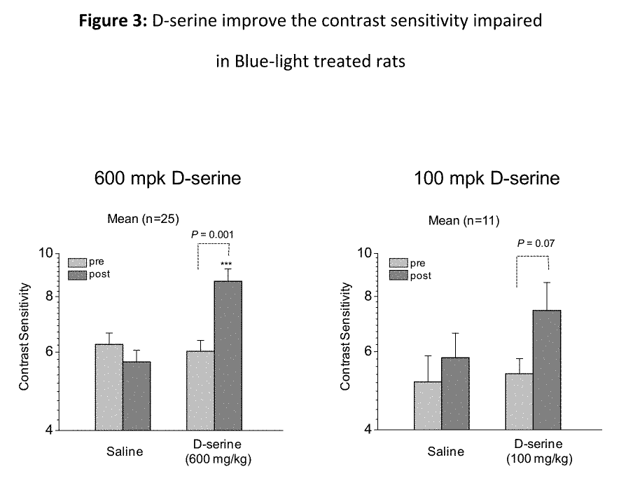

Two doses of D-serine were tested and vehicle (saline) was used as a control. Half of the rats were injected with either 100 or 600 mg/kg D-serine and the other half with saline in the first test; cross-over exposure took place one week later; the spatial frequencies were fixed at 0.575 cycles per degree (cpd). In FIG. 3, the mean values of contrast sensitivity are shown for rats treated with D-serine or saline, with a baseline taken prior to treatment (“pre”) and the contrast sensitivity measured 30 minutes after treatment (“post”). D-serine showed a dose-dependent improvement in contrast sensitivity in this model of retinal degenerative disease.

These findings provide the first evidence that exogenous D-serine enhances contrast sensitivity and visual function in a model of retinal system disorder or progressive degeneration. Other co-agonists of the NMDA receptor, such as those listed above are expected to have similar effects. D-serine and other NMDA receptor co-agonists are expected to have therapeutic benefits in age related deficits and neurological disorders which could lead to impairment in visual function. These include schizophrenia, Alzheimer's disease, degenerative diseases of the retina, such as glaucoma, wet ARMD, geographic atrophy, dry ARMD, optic neuritis, diabetic retinopathy, retinitis pigmentosa, rod dystrophies, cone dystrophies and other retinopathies, and amblyopia. D-serine and other NMDA receptor co-agonists are expected to have therapeutic benefits for patients, whose contrast sensitivities are reduced due to a side effect of laser vision surgery procedures, including LASIK and PRK. These compounds can be administered by any systemic route, or locally into the vitreous humor, and pro-drugs of amino acid analogs are anticipated to be effective.

### General Procedures Followed in Obtaining Experimental Data

LTP in Visual Cortex Slice: Following decapitation of the rat, the brain was rapidly removed and immersed in ice-cold artificial cerebrospinal fluid (ACSF) containing 124 mM NaCl, 3 mM KCl, 1.25 mM KH₂PO₄, 3.4 mM CaCl₂, 2.5 mM MgSO₄, 26 mM NaHCO₃, and 10 mM D-glucose. A block of visual cortex was created by removing the frontal 2/3 portion of the brain and the cerebellum. Coronal visual cortex slices of 375 μm were prepared from adult Sprague Dawley (SD) rats using a vibratome (VT 1000 S, Leica). The slices were maintained in an interface recording chamber perfused with preheated ACSF. Slices were continuously perfused with this solution at a rate of 1.00-1.50 ml/min while the surface of the slices was exposed to warm, humidified 95% O₂/5% CO₂ and maintained at 31±1° C. Visual cortex slices were allowed to recover for 1 hr before recording began. A single stimulating and recording electrode were placed in layer IV and III, respectively, to generate and record a field excitatory postsynaptic potentials (fEPSPs). Pulses were administered every 20 s using a current that produced a fEPSP that was 50% of the maximum spike free response. An input-output (IO) curve was done to determine the stimulation needed to achieve a stable baseline. Following a 15 min stable baseline recording period, a train of 5 theta bursts (each burst containing four pulses at 100 Hz with an inter-burst interval of 200 ms) were delivered to the slice. This was repeated 2 additional times with a 1 minute intertrain interval, and the level of LTP was recorded for at least 30 min. Changes in amplitude of the synaptic response were used to measure the extent of LTP because it was determined to be the more consistent parameter than the slope of the response. Control LTP values were obtained from slices not treated with drug. Different slices were used to study drug effects on LTP. After a 15 min baseline recording period, the compounds of interest were infused for 15 minutes followed by LTP induction. Washout of the compounds began 5 minutes after tetanization. Recording of the amplitude before, during, and after drug infusion was done. Statistical comparisons were done with LTP values recorded at 30 minutes after induction.

Sweep visually evoked potential (sweep VEP, sVEP), which was first introduced by Regan [1] in 1973, has become an important technique to measure visual function (VF). It is an objective method that can be used to assess contrast sensitivity (CS) in infants, young children and people with special needs. It was adapted to measure CS in animals.

VEP recording: The recording electrodes were permanently implanted into the right visual cortex of Long Evans rats at lambda and 4.5 mm lateral to the midline, to a depth of 800 microns (layer III/IV). A reference electrode was placed epidurally on the midline 1.2 mm anterior to bregma. All recordings were conducted in awake rats starting at least two weeks after recovery from surgery. During recording the rats were alert and restrained in a home-made restrainer. They were habituated 2-3 times pre-surgery and at least three more times during seven days post-surgery. PowerDiva software from Anthony Norcia (Smith Kettlewell Institute of Visual Sciences) was used for data acquisition and analysis.

Visual stimuli: Stimuli were presented on a CRT computer monitor and consist of full-field sine-wave gratings at fixed special frequency (0.5, 0.575 or 0.65 cpd in FIG. 1 and 0.575 cpd in FIG. 2), reversing at 6.25 Hz. VEPs were elicited by horizontally oriented gratings. The display was positioned 24 cm in front of the rat and centered at the vertical meridian. Mean luminance was held constant at 20 cd. One stimulus presentation (one trial) consists of a contrast sweep increasing from 2.5 to 70% in 15 log steps. A total of 20 to 30 trials were collected. Contrast thresholds (CT) were estimated using PowerDiva software. Contrast sensitivity (CS) is calculated as 1/CT.

1. A method for the treatment of amblyopia, the method comprising administering to a subject in need thereof an ophthalmically acceptable pharmaceutical composition containing a therapeutically effective amount of one or combination of co-agonists at the NMDA receptor.

2. A method according to claim 1, wherein the co-agonist at the NMDA receptor is selected from: Glycine, D-Serine, D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966 and 1-amino-1-carboxycyclobutane.

3. A method according to claim 1, wherein the co-agonist is D-serine.

4. A method for the treatment of visual system disorders caused by a deficit in N-methyl-D-aspartate receptor function, the method comprising administering to a subject in need thereof an ophthalmically acceptable pharmaceutical composition containing a therapeutically effective amount one or combination of co-agonists at the NMDA receptor.

5. A method according to claim 4, wherein the co-agonist at the NMDA receptor is selected from: Glycine, D-Serine, D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966 and 1-amino-1-carboxycyclobutane.

6. A method according to claim 4, wherein the co-agonist at the NMDA receptor is D-Serine.

7. A pharmaceutical composition comprising as active ingredient a therapeutically effective amount of co-agonist at the NMDA receptor and a pharmaceutically acceptable adjuvant, diluents or carrier, in methods for the treatment of visual system disorders.

8. A pharmaceutical composition according to claim 7, wherein the active ingredient is co-agonist at the NMDA receptor selected from: Glycine, D-Serine, D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966 and 1-amino-1-carboxycyclobutane.

9. A pharmaceutical composition according to claim 8, wherein the active ingredient is from: Glycine, D-Serine, D-Alanine, beta-fluoro-D-alanine, 1-aminocyclopropane-1-carboxylate, D-Cycloserine, 3-Amino-1-hydroxypyrrolid-2-one (HA-966), 4-methyl-HA-966 and 1-amino-1-carboxycyclobutane.

10. A pharmaceutical composition according to claim 7, wherein the active ingredient is D-serine.
